(19) United States
(12) Patent Application Publication
(10) Pub. No.: US 2025/0302293 A1
(43) Pub. Date: Oct. 2, 2025

(54) FACILITATING IOL ALIGNMENT USING AUTOMATED DETECTION OF PURKINJE IMAGES

(71) Applicant: Alcon Inc., Fribourg (CH)

(72) Inventors: Vignesh SURESH, Woodland Hills, CA (US); Lu YIN, Keller, TX (US); Ramesh SARANGAPANI, Coppell, TX (US)

(21) Appl. No.: 19/064,024

(22) Filed: Feb. 26, 2025

Related U.S. Application Data
(60) Provisional application No. 63/570,407, filed on Mar. 27, 2024.

(51) Int. Cl. A61B 3/00; A61B 3/13; A61B 3/14; A61F 2/16; G06T 7/00; G06T 7/10; G06T 7/73

(52) U.S. Cl. CPC A61B 3/0025; A61B 3/13; A61B 3/14; A61F 2/16; G06T 7/0016; G06T 7/10; G06T 7/74; G06T 2207/10056; G06T 2207/20081; G06T 2207/20084; G06T 2207/30041

(57) ABSTRACT

A system includes an ophthalmic microscope including a camera and a controller coupled to the camera. The controller configured to receive at least one image from the camera, the at least one image including a representation of an eye of a patient. The controller segments the at least one image using a machine learning model to obtain at least one segmented image including one or more labels of one or more Purkinje images represented in the at least one image. The controller produces an output according to the at least one segmented image. The output may be an estimate location of the visual axis of the eye. The output may include an estimate of an offset between the visual axis and a center of an IOL implanted in the eye. The segmented image may further label the IOL and possibly one or more items of anatomy of the eye.

### CROSS-REFERENCE TO RELATED APPLICATIONS

This application claims priority to U.S. Provisional Application No. 63/570,407, filed on Mar. 27, 2024, which is hereby incorporated by reference in its entirety.

### INTRODUCTION

The present disclosure relates generally to providing imaging during ophthalmic surgery, such as cataract surgery.

The human eye receives light through a clear outer portion called the cornea and focuses the resulting image by way of an ocular crystalline lens onto the retina. The quality of the focused image depends on many factors including the size and shape of the eye, and the transparency of the cornea and lens. When age or disease causes the lens to become less transparent, vision deteriorates because of the diminished light that is transmitted to the retina. This deficiency in the lens of the eye is medically known as a cataract. In addition, the crystalline lens may lose accommodation skills with age, which is called presbyopia. An accepted treatment for those conditions is the surgical removal of the crystalline lens followed by a replacement by an artificial intraocular lens (IOL).

It would be an advancement in the art to facilitate the performance of cataract surgery and other treatments.

### SUMMARY

In certain embodiments, a system includes an ophthalmic microscope including at least one camera and a controller coupled to the at least one camera. The controller configured to receive at least one image from the at least one camera, the at least one image including a representation of an eye of a patient. The controller segments the at least one image using a machine learning model to obtain at least one segmented image including one or more labels of one or more Purkinje images represented in the at least one image. The controller produces an output according to the at least one segmented image.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example ophthalmic system with which ophthalmic treatments are performed in an operating environment, in accordance with certain embodiments.

FIG. 2A is illustrates components of an ophthalmic microscope and Purkinje images and in accordance with certain embodiments.

FIG. 2B illustrates an arrangement of lights of an ophthalmic microscope.

FIG. 2C illustrates the reflection of Purkinje images from an intraocular lens (IOL) in accordance with certain embodiments.

FIG. 3 illustrates an example IOL.

FIG. 4 illustrates an IOL implanted in an eye and reflected Purkinje images that may be detected in accordance with certain embodiments.

FIG. 5 illustrates a system for estimating offset of an IOL from the visual axis of an eye in accordance with certain embodiments.

FIG. 6 is a process flow diagram of a method for evaluating alignment of an IOL with the visual axis of an eye in accordance with certain embodiments.

FIG. 7 illustrates an example computing device that implements, at least partly, one or more functionalities for facilitating visualization during ophthalmic surgery in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

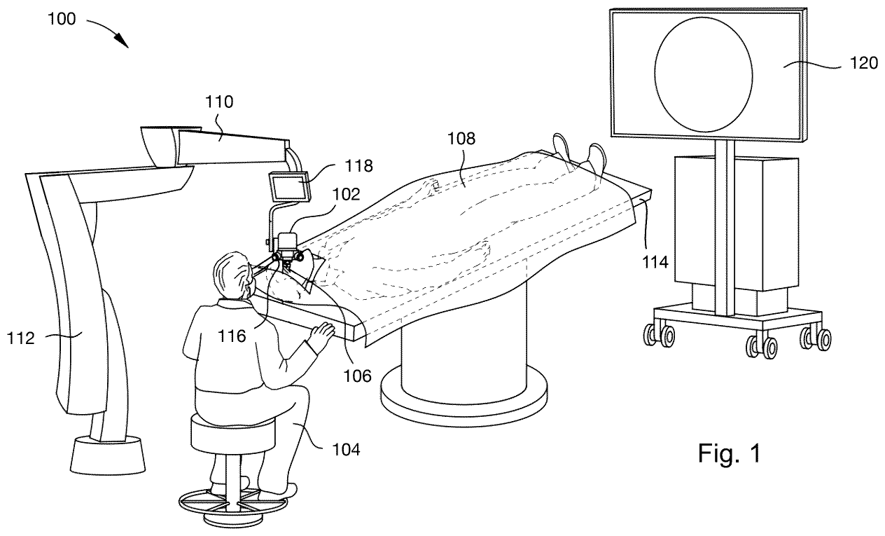

FIG. 1 illustrates an example ophthalmic system 100 with which ophthalmic treatments are performed in an operating environment. The system 100 includes an ophthalmic microscope 102. A surgeon 104 uses the ophthalmic microscope 102 to visualize structures on and in an eye 106 of a medical patient 108 in the field of view of the ophthalmic microscope 102. The ophthalmic microscope 102 is supported on, in this illustration, an adjustable overhead arm 110 of a microscope support pedestal 112. The patient 108 may be supported on an operating table 114. The ophthalmic microscope 102 is movable with the overhead arm 110 in three dimensions so that the surgeon 104 can position the ophthalmic microscope 102 as desired with respect to the eye 106 of the patient 108.

In certain embodiments, the ophthalmic microscope 102 comprises a high resolution, high contrast stereo viewing surgical microscope. The ophthalmic microscope 102 will often include a monocular eyepiece 116 or binocular eyepieces 116, through which the surgeon 104 will have an optically magnified view of the relevant eye structures that the surgeon 104 will need to see to accomplish a given surgery or diagnose an eye condition of the patient 108.

The ophthalmic microscope 102 includes a digital camera and broadband light source for capturing color (red, green, and blue) images, a multi-spectral imaging (MSI) device, and/or other type of imaging device. Digital images captured using the camera may be displayed on a display device within the ophthalmic microscope 102.

The ophthalmic microscope 102 may include two display devices, which are viewable through binocular eyepieces 116 and display images of the patient's eye 106 that are captured from different viewpoints by two cameras to provide stereoscopic viewing. For example, the ophthalmic microscope 102 may be implemented as the NGENUITY 3D VISUALIZATION SYSTEM provided by Alcon Inc. of Fort Worth Texas.

Images from the ophthalmic microscope 102 may additionally or alternatively be displayed on one or more display devices in the operating environment. For example, the one or more display devices may include a display device 118 fastened to the supporting arm 110 above the ophthalmic microscope 102.

In order to relieve the surgeon 104 from the need to constantly look into the eye pieces 116 to obtain a stereoscopic view, the one or more display devices may also include a display device 120 that can be implemented as a three-dimensional display device. The display device 120 may therefore provide a stereoscopic view of images captured using the ophthalmic microscope 102. The display device 120 may be embodied as any type of three-dimensional display device known in the art, including those that do or do not use special filtering glasses. For some types of three-dimensional display devices, the perception of three dimensions requires that the distance of the viewer from the display device 120 be within a threshold distance from the display device. The display device 120 may be mounted to a cart, a manually adjustable or robotic arm, or other manually or automatically adjustable support.

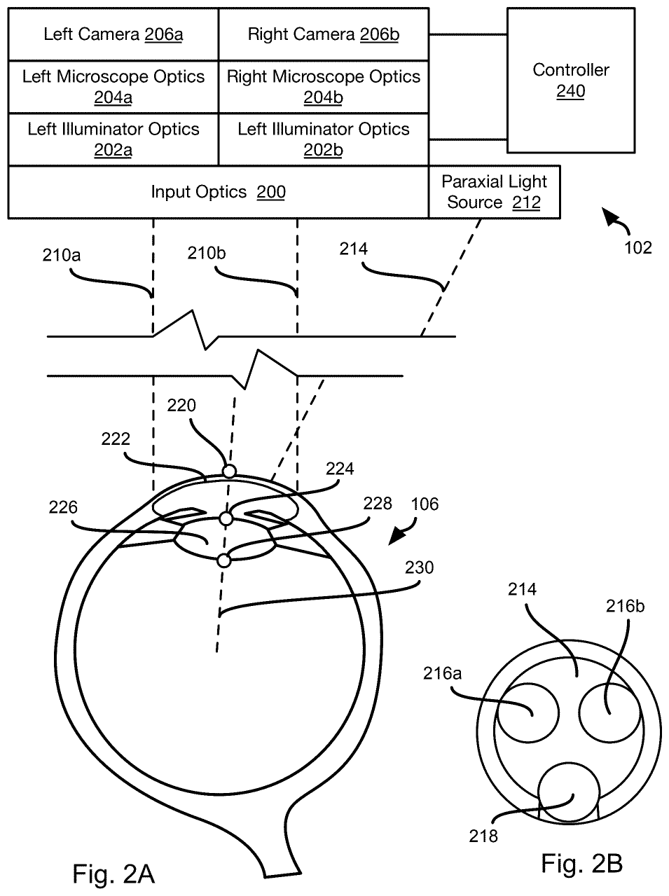

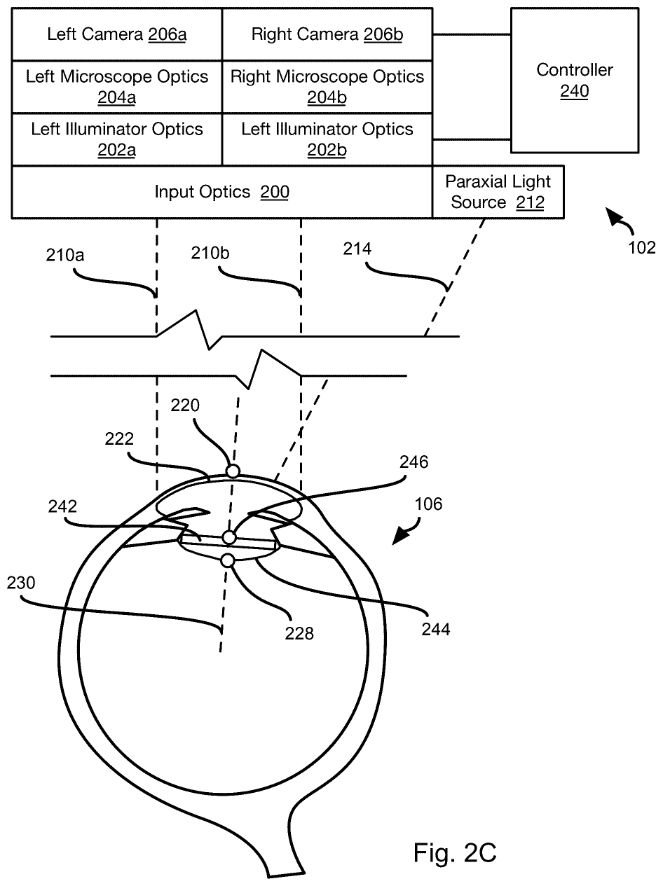

FIG. 2A is schematic diagram of the ophthalmic microscope 102, which includes input optics 200, left and right illuminator optics 202a, 202b, left and right microscope optics 204a, 204b, and left and right eye cameras 206a, 206b. As used herein, “left” and “right” are used to refer to first and second instances of a components facilitating visualization by the left and right eyes of the surgeon 104. The use of “left” and “right” shall be understood as exemplary only and it shall be understood that these can be readily interchanged without change in functionality.

The input optics 200 receive light reflected from the eye 106 of the patient. The input optics 200 may include a set of lenses with a common optical axis or two sets of lenses with offset and/or non-parallel optical axes, e.g., right and left sets of lenses. The left and right illuminator optics 202a, 202b include light sources and optics that both of (a) direct light from the light sources onto the eye 106 and (b) permit light reflected from the eye to pass through the illuminator optics 202a, 202b. The left and right illuminator optics 202a, 202b may therefore each include a beam splitter and possibly one or more lenses to facilitate this function. The light sources of the left and right illuminator optics 202a, 202b may be embodied as light emitting diodes (LED) or other light sources. The light sources may be operable at a variety of intensities and colors. In certain embodiments, the light sources may include LEDs having three different wavelength distributions (e.g., centered on red, green, and blue wavelengths) and having independently selectable intensities such that the color emitted by the light source may be controlled. The light sources may additionally or alternatively include infrared or near-infrared light sources enabling one or both of (a) illumination using light that is not visible to the patient and (b) illumination using visible light that causes very little patient discomfort.

Light reflected from the eye 106 passes through the left and right illuminator optics 202a, 202b and is magnified by left and right microscope optics 204a, 204b, respectively. The magnification of the left and right microscope optics 204a, 204b may be adjustable. Likewise, the depth of focus of the left and right microscope optics 204a, 204b may be adjustable. Light output reflected from the eye is emitted by the left and right microscope optics 204a, 204b onto left and right cameras 206a, 206b. The left and right cameras 206a, 206b output images that may be displayed on any of the display devices 118, 120, an internal display of the ophthalmic microscope 102, or other display device. The images output by the left and right cameras 206a, 206b may further be processed according to the methods described herein to evaluate alignment of an IOL with respect to the eye 106.

In some embodiments, only one camera is used (referred to herein as the “single microscope camera”). In such embodiments, there may be a single set of microscope optics and a single set of illuminator optics. Alternatively, the illustrated pairs of left and right microscope optics 204a, 204b and left and right illuminator optics 202a, 202b may be used.

Light from the illuminator optics 202a, 202b is transmitted onto the eye 106. The light is directed along the optical axes 210a, 210b of the left and right sides of the ophthalmic microscope 102, i.e., the optical axes 210a, 210b of the left and right illuminator optics 202a, 202b. The optical axes 210a, 210b may be parallel to one another or converge at a point outward from the input optics 200. In some embodiments, additional illumination is provided by a paraxial light source 212 directed along axis 214 that is non-parallel with respect to the optical axes 210a, 210b, such as at an angle of between 5 and 12 degrees. As used herein, the light from the left and right illuminator optics 202a, 202b is referred to as “left coaxial light,” “right coaxial light,” or collectively as “coaxial lights.” The light from the paraxial light source 212 is referred to as “paraxial light.”

FIG. 2B illustrates an example appearance of the ophthalmic microscope to the eye 106 of the patient. The light emitted by the left and right illuminator optics 202a, 202b appears as two bright spots 216a, 216b and the light from the paraxial light source 212 appears as a third bright spot 218 offset from the bright spots 216a, 216b.

Turning back to FIG. 2A, the light from the left and right illuminator optics 202a, 202b and the paraxial light source 212 are incident on the eye 106 and portions thereof reflect off of different surfaces of the eye 106. For example, a portion 220 of the light reflects from the anterior surface of the cornea 222 and is referred to as the P1 Purkinje image. A portion also reflects from the posterior surface of the cornea and is referred to as the P2 Purkinje image but is less discernable and is typically not used. A portion 224 of the light reflects from the anterior surface of the crystalline lens 226 and is referred to as the P3 Purkinje image. A portion 228 of the light reflects from the posterior surface of the crystalline lens 226 and is referred to as the P4 Purkinje image. In practice, the P1 and P4 Purkinje images are the most visible and used clinically.

A controller 240 may be coupled to the left and right cameras 206a, 206b to receive images output by the left and right cameras 206a, 206b. The controller 240 may be implemented by a computing system 700 as described below. The controller 240 may also be coupled to the left and right illuminator optics 202a, 202b and the paraxial light source 212 to control the operation thereof.

Referring to FIG. 2C, an IOL 242 may be implanted within the capsular bag 244 that formerly contained the lens 226. The anterior surface of the capsular bag 244 and the lens 226 will have been previously removed. A portion 246 of light reflected from the anterior surface of the IOL 242 likewise creates a Purkinje image, sometimes referred to as the P1 Purkinje image of a pseudophakic eye (eye with an artificial lens).

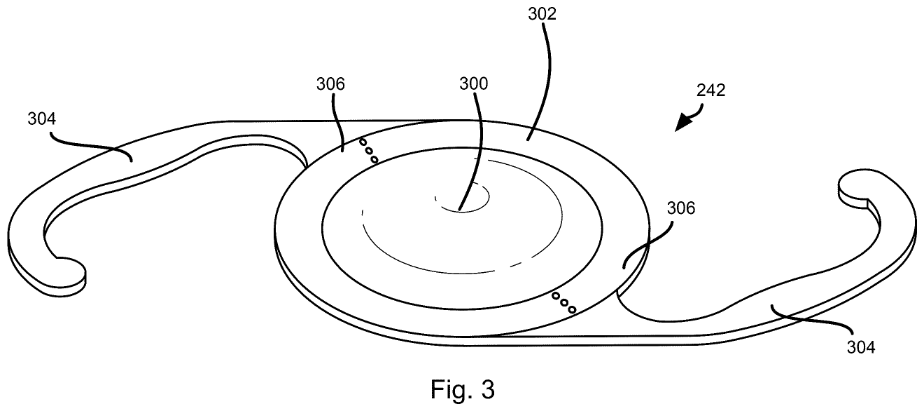

Referring to FIG. 3, the IOL 242 may be embodied as the illustrated toric IOL. The IOL 242 may include a lens portion 300 including surfaces defining a lens. The lens portion 300 may be a refractive lens that relies on curvature of anterior and/or posterior surfaces. The lens portion 300 may additionally or alternatively include diffractive elements, such as concentric refractive rings providing multi-focal vision.

The IOL 242 may include nothing more than a lens portion 300, and possibly a perimeter portion 302 for engaging the capsular bag 244. Alternatively, the IOL 242 may include haptics 304 secured to the perimeter portion. The haptics 304 operate as springs pushing outwardly against the capsular bag 244 in order to stabilize the IOL 242 and/or maintain the orientation of the IOL 242 within the capsular bag 244 about the visual axis 230. The lens portion 300 and/or perimeter portion 302 may include markings 306, e.g., fiducial markings, that may be used to determine the orientation of the IOL 242 about the visual axis 230 to facilitate correction of astigmatism.

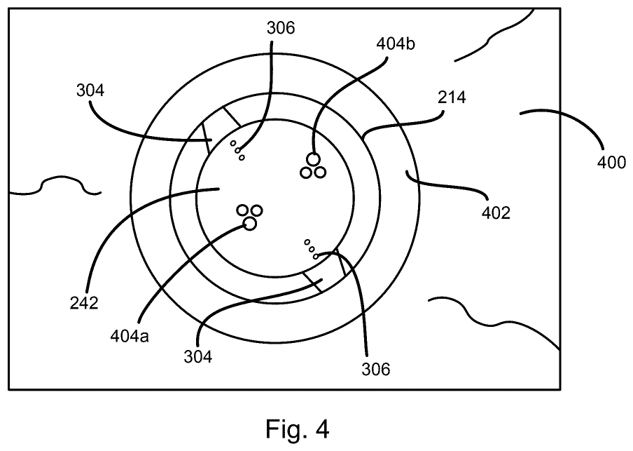

FIG. 4 illustrates an example image that may be captured using the left and right cameras 206a, 206b or the single microscope camera. The image may show the sclera 400 and iris 402 of the eye 106. Both the sclera 400 and iris 402 may include distinguishing features that enable the image to be registered with respect to known anatomy of the eye, such as recorded in a reference image that is part of a treatment plan. The image further shows the IOL 242 with the marks 306 being visible. In some instances, some portion of the haptics 304 may also be visible. The IOL 242 is made of transparent material such that actual visibility will be much less than that shown.

The image further includes one or more Purkinje images 404a, 404b. As is apparent the Purkinje images 404a, 404b may be flipped relative to one another, such as due to Purkinje image 404a being a reflection from the anterior surface of the IOL 242 and Purkinje image 404b being a reflection from the posterior surface of the capsular bag 244, i.e., a reflection of light has passed through the IOL 242 and been flipped thereby.

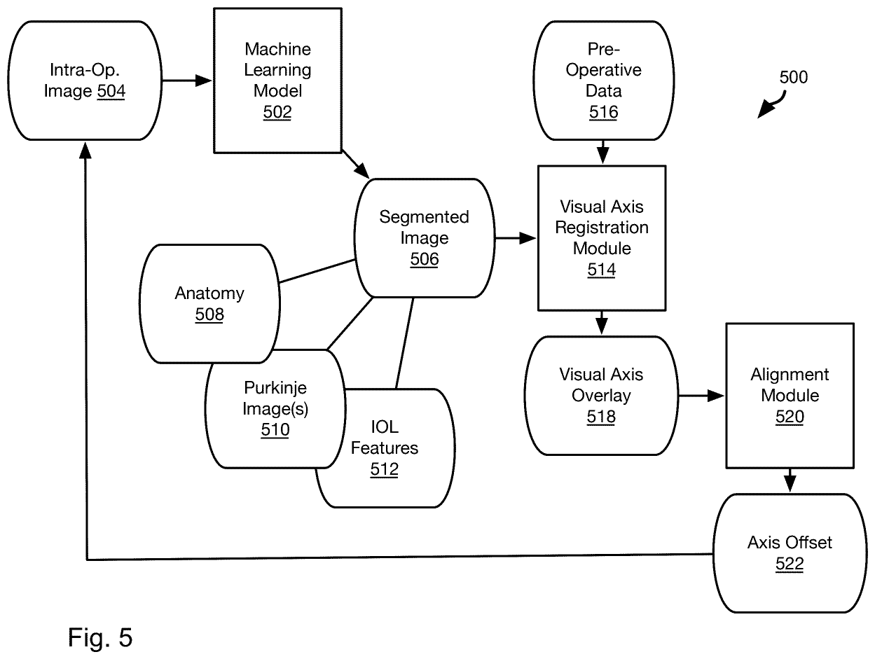

FIG. 5 illustrates a system 500 that may be implemented with respect to images received from the left and right cameras 206a, 206b or the single microscope camera. In the following example, a single time series of images is described as “the intra-operative images” from “the camera,” with the understanding that “the camera” could be either of the left and right cameras 206a, 206b or the single microscope camera. Where both the left and right cameras 206a, 206b are used, results of processing intra-operative images for the same time step (e.g., captured within 10 milliseconds of one another) according to the system 500 may be combined or used separately as pointed out below.

The system 500 includes a machine learning model 502. The machine learning model 502 may include one or more of a neural network (NN), convolution neural network (CNN), or other type of artificial intelligence model. For example, the machine learning model 502 may be include one or more of a U-NET machine learning model, You Only Look Once (YOLO) machine learning model, or another available machine learning model that can be trained to perform the tasks ascribed herein to the machine learning model 502.

In particular, the machine learning model 502 may be trained to receive an intra-operative image 504 and output a segmented image 506. The segmented image 506 may include labels 508 of pixels of the intra-operative image 504 corresponding to anatomy (e.g., the sclera 400, iris 402, limbus, or other items of anatomy), labels 510 of pixels of the intra-operative image 504 corresponding to Purkinje images (e.g., Purkinje images 404a, 404b), and labels 512 of IOL features (e.g., haptics 304, marks 306, edge of the peripheral portion 302, edge of the lens portion 300, diffractive rings, etc.).

The machine learning model 502 may include multiple machine learning models, each being trained to generate one type of label of the labels 508, 510, 512. In either case, the machine learning model 502 may be trained using training data entries, each training data entry including a training intra-operative image and one or more human-generated labels, such as any of the labels 508, 510, 512. The machine learning model 502 may process the training intra-operative image and produce one or more estimated labels, which may then be compared to the one or more human-generated labels and the machine learning model 502 may then be updated according to the comparison.

The segmented image 506, which may include the intra-operative image and one or more of the labels 508, 510, 512, may be processed by a visual axis registration module 514, which produces a visual axis overlay 518. The visual axis registration module 514 may further take, as an input, one or more items of pre-operative data 516. For example, the pre-operative data 516 may include a reference image of the eye 106 and labels applied to the reference image, such as a label of the visual axis, labels of one or more items of anatomy (e.g., any of the anatomy identified by label 58), a label of an axis defining a desired orientation of the IOL 242 (e.g., a line intersecting the marks 306). The pre-operative data may include depth information, e.g., depth of the posterior surface of the capsular bag 244 relative to the apex of the cornea, depth of the iris plane, or depth of other items of anatomy along the visual axis 230.

The visual axis registration module 514 estimates a location of the visual axis 230 in the intra-operative image 504. Since the intra-operative image is a two-dimensional image and the visual axis 230 is defined in three-dimensional space, the location of the visual axis as determined by the visual axis registration module 514 may be an estimated point in the intra-operative image 504 representing a point along the visual axis that is of relevance to the surgeon, such as a point along the visual axis within the capsular bag 244 (e.g., at a predefined offset from the posterior surface of the capsular bag 244 and the apex of the cornea as defined in the pre-operative data).

For example, the labels 510 of the Purkinje images may be used to determine an estimated location of the visual axis. If multiple Purkinje images are aligned in the intra-operative image 504, then the visual axis may be estimated to be at the center of the Purkinje images. If the Purkinje images are not aligned, the visual axis may be inferred to be at the midpoint of a line connecting the Purkinje images. The pre-operative data 516 may be used to infer the location of the visual axis. For example, using a reference image included in the pre-operative data 516, the location of the visual axis labeled in the reference image may be mapped to a location on the pre-operative image. The locations of the Purkinje images (i.e., magnitude and direction of misalignment) may then be used to infer tilt of the eye 106 relative to the ophthalmic microscope 102 and/or relative to the orientation of the eye 106 when the reference image was captured. The visual axis as determined using the reference image may then be adjusted in correspondence with the inferred tilt to obtain the estimated visual axis. Adjusting the estimated visual axis with reference to tilt may take into account depth information included in the pre-operative data, e.g., a degree of translation as a function of the tilt (angle) and the depth information.

The result of the processing of the visual axis registration module 514 may be a visual axis overlay 518. The visual axis overlay 518 may be a label (e.g., pixel position) of the estimated location of the visual axis as defined above. The visual axis overlay 518 may be input to an alignment module 520. The alignment module 520 estimates a degree of misalignment between the estimated visual axis and a center of the IOL 242. The alignment module 520 may, for example, identify the center of the IOL 242 by evaluating the labels 512 of IOL features. For example, the alignment module 520, may identify the center of a blob of pixels labeled as corresponding to the lens portion 300 as the center of the IOL 242. In another example, the alignment module 520 may determine the center of the IOL 242 to be the center of pixels representing a combination of the lens portion 300 and peripheral portion 302. In another example, the alignment module 520 may determine the center of the IOL 242 as being a center point between the marks 306. In another example, the alignment module 520 may also determine the center of the IOL 242 by performing some other evaluation of the labels 512.

The alignment module 520 may then output an axis offset 522, e.g., a representation of a difference between the estimated location of the visual axis and the center of the IOL. The representation may be an offset (estimated X and Y offset distances), a graphical indication of the degree of misalignment (e.g., an arrow showing a direction to move the IOL in order to achieve alignment), or other representation. In the event that the difference is below a threshold, the axis offset 522 may include an indicator that no further alignment is needed.

The system 500 may be modified to incorporate additional functionality where the intra-operative images 504 (hereinafter “right and left intra-operative images”) are received simultaneously (e.g., within 10 milliseconds) from left and right cameras 206a, 206b. For example, for each type of label 508, 510, 512, a three-dimensional volumetric label may be created using labels derived from the right and left intra-operative images. For example, a label 510 of a Purkinje image for the left intra-operative image and a label 510 of the same Purkinje image in the right intra-operative image may be evaluated using stereoscopic vision techniques to obtain a three-dimensional representation of the Purkinje image, e.g., a volumetric image. Each item of anatomy represented in the labels 508 and each IOL feature represented in the labels 512 may be processed in a like manner to obtain a volumetric image. The three-dimensional location of the center of the IOL 242 may be estimated from the volumetric image obtained from the labels 512 of IOL features. The three-dimensional locations of the Purkinje images may be used to infer a location of the visual axis (or a point along the visual axis as defined above) in three-dimensions. The three-dimensional positions of the visual axis and center of the IOL 242, or some other reference point on the IOL 242, may then be compared to estimate alignment.

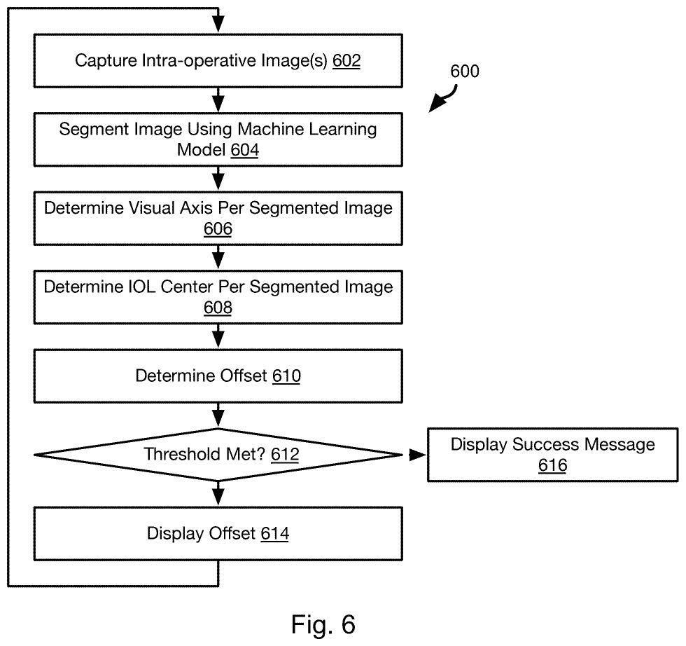

FIG. 6 illustrates a method 600 that may be performed with the controller 240, such as a controller 240 configured to implement the system 500. The method 600 may be performed repeatedly, such as for each frame (e.g., each time step), or every Nth frame (N being an integer greater than 1) of a video stream output by the left and right cameras 206a, 206b or the single microscope camera.

At step 602 one or more intra-operative images are captured and, at step 604, each of the one or more intra-operative images is segmented using the machine learning model 502 to obtain a segmented image, such as a segmented image including some or all of the labels 508, 510, 512 as defined above. An estimated location of the visual axis is then determined at step 606, based on the segmented inter-operative images, such as using the approach described above with respect to the functionality of the visual axis registration module 514. At step 608, the center of the IOL 242 is determined based on the segmented image and, at step 610, an offset between the center of the IOL 242 and the estimated location of the visual axis is determined, e.g., an amount of separation between the center of the IOL 242 and the estimated location of the visual axis. Steps 608 and 610 may be performed by implementing the functionality of the alignment module 520 as described above.

At step 612, the offset is evaluated with respect to a threshold condition. If the threshold condition is not met, then a representation of the offset is displayed at step 614. For example, step 614, may include displaying any of the representations of the axis offset 522 as described above. The representation of the offset may be displayed on the display device 120, the display device 120, a display device internal to the ophthalmic microscope 102, or some other display device. If the threshold condition is met, then a success message may be displayed at step 616. The success message may be text, a symbol, color, or other visual attributes indicating that further adjustment of the IOL 242 is not required. The threshold condition may be the offset being below a maximum permissible offset.

Steps 612 and either step 614 or step 616 may be performed substantially in real time, such as within 10, 5, 4, 2, or 1 time step of when the intraoperative image was captured at step 602 for a given iteration of the method 600 in which step 612 is performed.

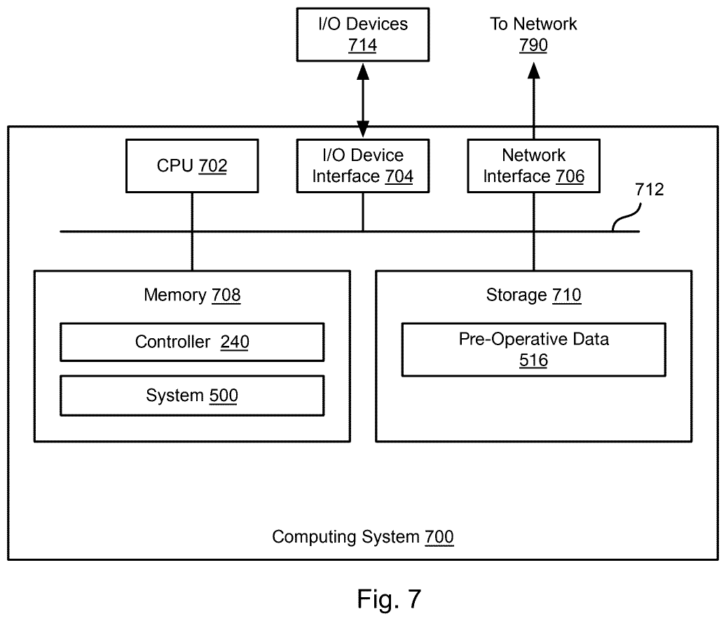

FIG. 7 illustrates an example computing system 700. The ophthalmic microscope 102 and the display device 120 may incorporate a computing device having some or all of the attributes of the computing system 700.

As shown, computing system 700 includes a central processing unit (CPU) 702, one or more I/O device interfaces 704, which may allow for the connection of various I/O devices 714 (e.g., keyboards, displays, mouse devices, pen input, etc.) to computing system 700, network interface 706 through which computing system 700 is connected to network 790, a memory 708, storage 710, and an interconnect 712.

CPU 702 may retrieve and execute programming instructions stored in the memory 708. Similarly, CPU 702 may retrieve and store application data residing in the memory 708. The interconnect 712 transmits programming instructions and application data, among CPU 702, I/O device interface 704, network interface 706, memory 708, and storage 710. CPU 702 is included to be representative of a single CPU, multiple CPUs, a single CPU having multiple processing cores, and the like.

Memory 708 is representative of a volatile memory, such as a random access memory, and/or a nonvolatile memory, such as nonvolatile random access memory, phase change random access memory, or the like. As shown, memory 708 may store executable code implementing one or both of the controller 240 and the system 500.

Storage 710 may be non-volatile memory, such as a disk drive, solid state drive, or a collection of storage devices distributed across multiple storage systems. Storage 710 may optionally store the pre-operative data 516.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. An ophthalmic visualization system comprising:
an ophthalmic microscope including at least one camera configured to capture at least one image including a representation of an eye of a patient positioned in proximity to the ophthalmic microscope; and
a controller coupled to the at least one camera, the controller configured to:
receive the at least one image from the at least one camera;
segment the at least one image using a machine learning model to obtain at least one segmented image including one or more labels of one or more Purkinje images represented in the at least one image; and
produce an output according to the at least one segmented image.

2. The ophthalmic visualization system of claim 1, wherein the controller is further configured to:
calculate an estimated position of a visual axis of the eye of the patient according to the one or more labels of the one or more Purkinje images; and
produce the output according to the estimated position of the visual axis.

3. The ophthalmic visualization system of claim 2, wherein the controller is further configured to calculate the estimated position of the visual axis of the eye using pre-operative data.

4. The ophthalmic visualization system of claim 3, wherein the pre-operative data comprises a reference image of the eye of the patient.

5. The ophthalmic visualization system of claim 2, wherein the at least one segmented image further includes one or more labels of an intraocular lens (IOL) positioned within the eye of the patient, the controller being further configured to:
calculate an estimated center of the IOL according to the one or more labels of the IOL;
calculate an offset between the estimated center of the IOL and the estimated position of the visual axis; and
produce the output according to the offset.

6. The ophthalmic visualization system of claim 5, wherein the controller is configured to:
evaluate the offset with respect to a threshold condition; and
if the offset meets the threshold condition, produce the output as a success message.

7. The ophthalmic visualization system of claim 5, wherein the controller is configured to:
evaluate the offset with respect to a threshold condition; and
if the offset does not meet the threshold condition, produce the output as a representation of the offset.

8. The ophthalmic visualization system of claim 1, wherein the machine learning model comprises at least one of a neural network (NN) or a convolution neural network (CNN).

9. The ophthalmic visualization system of claim 1, wherein the machine learning model comprises at least one of a U-NET or a You Only Look Once (YOLO) machine learning model.

10. The ophthalmic visualization system of claim 1, wherein the at least one camera comprises a left camera and a right camera and the at least one image comprises at least two images captured substantially simultaneously by the left camera and the right camera.

11. A method for ophthalmic visualization comprising:
receiving, by a controller, at least one image from at least one camera of an ophthalmic microscope having an eye of a patient in a field of view thereof;
segmenting, by the controller, the at least one image using a machine learning model to obtain at least one segmented image including one or more labels of one or more Purkinje images represented in the at least one image; and
producing, by the controller, an output according to the at least one segmented image.

12. The method of claim 11, further comprising:
calculating, by the controller, an estimated position of a visual axis of the eye of the patient according to the one or more labels of the one or more Purkinje images; and
producing, by the controller, the output according to the estimated position of the visual axis.

13. The method of claim 12, further comprising calculating, by the controller, the estimated position of the visual axis of the eye using pre-operative data.

14. The method of claim 13, wherein the pre-operative data comprises a reference image of the eye of the patient.

15. The method of claim 12, wherein the at least one segmented image further includes one or more labels of an intraocular lens (IOL) positioned within the eye of the patient, the method further comprising:
calculating, by the controller, an estimated center of the IOL according to the one or more labels of the IOL;
calculating, by the controller, an offset between the estimated center of the IOL and the estimated position of the visual axis; and
producing, by the controller, the output according to the offset.

16. The method of claim 15, further comprising:
evaluating, by the controller, the offset with respect to a threshold condition;
determining, by the controller, that the offset meets the threshold condition; and
in response to determining that the offset meets the threshold condition, outputting, by the controller, a success message.

17. The method of claim 15, further comprising:
evaluating, by the controller, the offset with respect to a threshold condition; and
determining, by the controller, that the offset does not meet the threshold condition;
in response to determining that the offset does not meet the threshold condition, produce the output as a representation of the offset.

18. The method of claim 11, wherein the machine learning model comprises at least one of a neural network (NN) or a convolution neural network (CNN).

19. The method of claim 11, wherein the machine learning model comprises at least one of a U-NET machine learning model or a You Only Look Once (YOLO) machine learning model.

20. The method of claim 11, wherein the at least one camera comprises a left camera and a right camera and the at least one image comprises at least two images captured substantially simultaneously by the left camera and the right camera.
